(19) United States
(12) Patent Application Publication
(10) Pub. No.: US 2024/0046183 A1
(43) Pub. Date: Feb. 8, 2024

(54) RECEIPT DATA EXAMINATION DEVICE, RECEIPT DATA EXAMINATION METHOD, AND STORAGE MEDIUM

(71) Applicant: NEC Corporation, Tokyo (JP)

(72) Inventors: Eiji YUMOTO, Tokyo (JP); Masahiro Kubo, Tokyo (JP); Kosuke Nishihara, Tokyo (JP)

(73) Assignee: NEC Corporation, Tokyo (JP)

(21) Appl. No.: 18/380,933

(22) Filed: Oct. 17, 2023

Related U.S. Application Data
(63) Continuation of application No. 18/282,034, filed on Jan. 1, 0001.

(51) Int. Cl. G06Q 10/0635; G16H 15/00

(52) U.S. Cl. CPC G06Q 10/0635; G16H 15/00

(57) ABSTRACT

Based on receipt data indicating a detailed statement of medical fees, receipt data conversion information indicating a presence or absence of a medical care information item that corresponds to a plurality of examination failure conditions is generated. A failure risk level based on the medical care information item included in the receipt data is calculated by using the receipt data conversion information and an examination failure risk predictor. A risk factor indicating a medical care information item with a degree of contribution to the failure risk level calculation that is greater than or equal to a prescribed threshold value for degree of contribution is specified. A revision screen for the medical care information item that is a risk factor when the failure risk level is greater than or equal to a prescribed threshold value for risk level is output.

### CROSS-REFERENCE TO RELATED APPLICATIONS

This application is a Continuation of U.S. application Ser. No. 18/282,034 filed on Sep. 14, 2023, which is a National Stage Entry of PCT/JP2021/012927 filed on Mar. 26, 2021, the contents of all of which are incorporated herein by reference, in their entirety.

### TECHNICAL FIELD

The present invention relates to a receipt data examination device, a receipt data examination method, and a storage medium.

### BACKGROUND ART

A medical care provider creates receipt data that shows details of medical fees and so forth and sends the receipt data to an examination institution, and can then receive the unreceived portion of the medical expenses other than the patient-liable portion of the medical fees if the examination passes. If there is a flaw in the receipt data, the examination will not pass and the unreceived portion of medical fees will not be received. Therefore, it is necessary to check whether or not the medical fee details included in receipt data pass the examination. Techniques relevant to this are disclosed in Patent Document 1 and Patent Document 2.

Patent Document 1 discloses a technique of referring to the amount of medical fees to specify the amount of medical fees that may not be paid to the medical care provider. Patent Document 2 discloses a technique that facilitates discovery of errors in each receipt.

### CITATION LIST

### Patent Documents

Patent Document 1: Japanese Unexamined Patent Application, First Publication No. 2014-21680

Patent Document 2: Japanese Unexamined Patent Application, First Publication No. 2004-265444

### SUMMARY OF THE INVENTION

### Problems to be Solved by the Invention

Incidentally, in addition to those cases of incorrect records such as recording errors among many records in receipt data that do not comply with the examination, in a case where there is a possibility that the examining party may consider the combination of medical care information items recorded in a receipt, such as the relationship between primary disease name and medical care method, to be inappropriate, then records that are not in fact incorrect may also not conform to the examination. Therefore, it was necessary to specify not only simple recording errors in a receipt, but also other records that may possibly be non-compliant with the examination.

Thus, an object of the present invention is to provide a receipt data examination device, a receipt data examination method, and a storage medium for solving the problem mentioned above.

### Means for Solving the Problem

According to a first example aspect of the present invention, a receipt data examination device includes: a conversion means that generates, based on receipt data indicating a detailed statement of medical fees, receipt data conversion information indicating a presence or absence of a medical care information item that corresponds to a plurality of examination failure conditions; a failure risk calculation means that calculates a failure risk level based on the medical care information item included in the receipt data, by using the receipt data conversion information and an examination failure risk predictor; a risk factor specification means that specifies a risk factor indicating a medical care information item with a degree of contribution to the failure risk level calculation that is greater than or equal to a prescribed threshold value for degree of contribution; and a revision processing means that outputs a revision screen for the medical care information item that is a risk factor when the failure risk level is greater than or equal to a prescribed threshold value for risk level.

According to a second example aspect of the present invention, a receipt data examination method includes: generating, based on receipt data indicating a detailed statement of medical fees, receipt data conversion information indicating a presence or absence of a medical care information item that corresponds to a plurality of examination failure conditions; calculating a failure risk level based on the medical care information item included in the receipt data, by using the receipt data conversion information and an examination failure risk predictor; specifying a risk factor indicating a medical care information item with a degree of contribution to the failure risk level calculation that is greater than or equal to a prescribed threshold value for degree of contribution; and outputting a revision screen for the medical care information item that is a risk factor when the failure risk level is greater than or equal to a prescribed threshold value for risk level.

According to a third example aspect of the present invention, a storage medium has stored therein a program that causes a computer of a receipt data examination device to function as: a conversion means that generates, based on receipt data indicating a detailed statement of medical fees, receipt data conversion information indicating a presence or absence of a medical care information item that corresponds to a plurality of examination failure conditions; a failure risk calculation means that calculates a failure risk level based on the medical care information item included in the receipt data, by using the receipt data conversion information and an examination failure risk predictor; a risk factor specification means that specifies a risk factor indicating a medical care information item with a degree of contribution to the failure risk level calculation that is greater than or equal to a prescribed threshold value for degree of contribution; and a revision processing means that outputs a revision screen for the medical care information item that is a risk factor when the failure risk level is greater than or equal to a prescribed threshold value for risk level.

### Effect of Invention

According to the present invention, it is possible to specify not only simple recording errors in a receipt, but also other records that may possibly be non-compliant with the examination according to past cases.

### BRIEF DESCRIPTION OF THE DRAWINGS

FIG. 1 is a diagram showing a schematic configuration of a medical care system according to the present example embodiment.

FIG. 2 is a hardware configuration diagram of a receipt data examination device according to the present example embodiment.

FIG. 3 is a function block diagram of the receipt data examination device according to a first example embodiment.

FIG. 4 is a first diagram showing a processing flow of the receipt data examination device according to the first example embodiment.

FIG. 5 is a second diagram showing a processing flow of the receipt data examination device according to the first example embodiment.

FIG. 6 is a function block diagram of a receipt data examination device according to a second example embodiment.

FIG. 7 is a first diagram showing a processing flow of the receipt data examination device according to the second example embodiment.

FIG. 8 is a second diagram showing a processing flow of the receipt data examination device according to the second example embodiment.

FIG. 9 is a diagram showing a minimum configuration of the receipt data examination device according to the present example embodiment.

FIG. 10 is a diagram showing a processing flow of the receipt data examination device of the minimum configuration.

### EXAMPLE EMBODIMENT

Hereinafter, a receipt data examination device according to an example embodiment of the present invention will be described, with reference to the drawings.

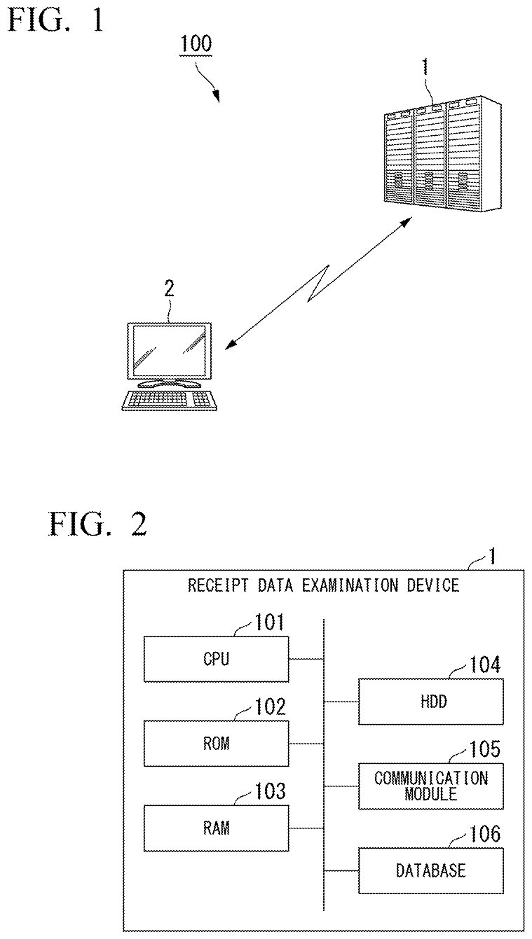

FIG. 1 is a diagram showing a schematic configuration of a medical care system including the receipt data examination device according to the present example embodiment.

As shown in FIG. 1, a medical care system 100 is configured such that a receipt data examination device 1 and a terminal 2 used by a user such as a medical care administration clerk are connected to each other via a wireless communication network or a wired communication network. The user uses the terminal 2 to create receipt data such as medical fee details, and instructs the receipt data examination device 1 to perform examination processing on the created receipt data. The receipt data examination device 1 performs the examination processing on the receipt data.

FIG. 2 is a hardware configuration diagram of the receipt data examination device.

As shown in this figure, the receipt data examination device 1 is a computer that includes hardware units such as a CPU (Central Processing Unit) 101, a ROM (Read Only Memory) 102, a RAM (Random Access Memory) 103, a HDD (Hard Disk Drive) 104, a communication module 105, and a database 106. The terminal 2 also has a similar hardware configuration.

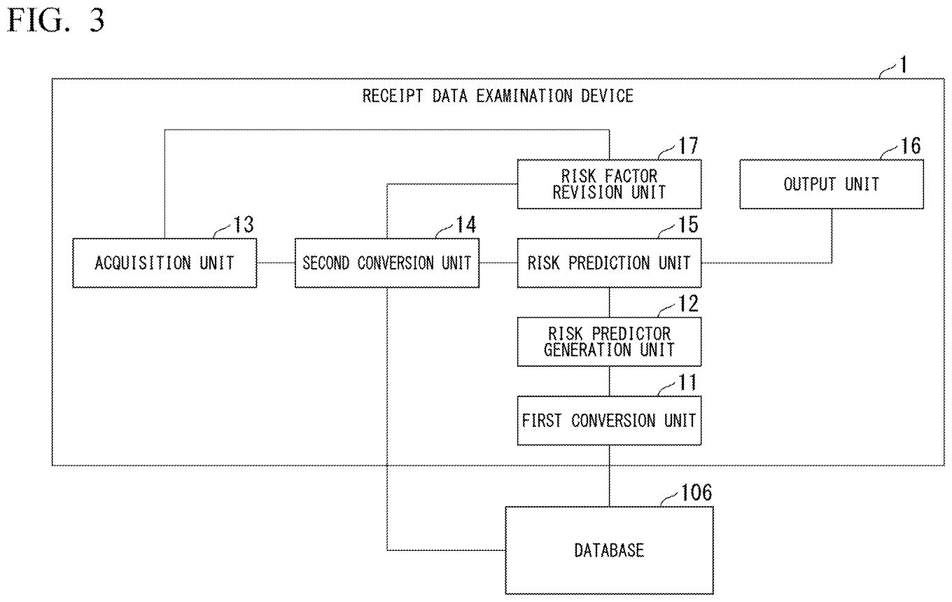

FIG. 3 is a function block diagram of the receipt data examination device according to the first example embodiment.

The receipt data examination device 1 executes an examination program that is preliminarily stored. As a result, the receipt data examination device 1 serves the functions of a first conversion unit 11, a risk predictor generation unit 12, an acquisition unit 13, a second conversion unit 14, a risk prediction unit 15, an output unit 16, and a risk factor revision unit 17.

The first conversion unit 11 generates receipt data conversion information that indicates the presence or absence of medical care information items corresponding to a plurality of examination failure conditions, using the receipt data recorded in the database 106. The medical care information items are, for example, pieces of information recorded in a receipt, such as primary disease name and medical care method.

The risk predictor generation unit 12 uses machine learning to perform learning processing on the relationship between receipt data conversion information generated from the receipt data stored in the past, an examination failure result of the receipt data, and the medical care information item that is a failure factor of the examination failure result, and generates an examination failure risk predictor.

The acquisition unit 13 acquires receipt data indicating a receipt to be examined and other information input to the receipt data examination device 1 by another user.

As with the first conversion unit 11, the second conversion unit 14 generates receipt data conversion information that indicates the presence or absence of medical care information items corresponding to a plurality of examination failure conditions, using the acquired examination target receipt data.

The risk prediction unit 15 uses the receipt data conversion information and the examination failure risk predictor to calculate a failure risk level based on the medical care information items or a combination thereof included in the receipt data. Moreover, the risk prediction unit 15 specifies a risk factor indicating a medical care information item with a degree of contribution to failure risk level calculation that is greater than or equal to a prescribed threshold value for degree of contribution.

The output unit 16 outputs, to a monitor or the like, an examination result screen showing the calculated failure risk level and the risk factor.

If the failure risk level is greater than or equal to a prescribed threshold value for risk level, the risk factor revision unit 17 revises the medical care information item that is a risk factor based on the user's instruction.

By serving such functions, the receipt data examination device 1 provides a technique that enables specification of not only simple recording errors in a receipt, but also other records that may possibly be non-compliant with the examination according to past cases.

### First Example Embodiment

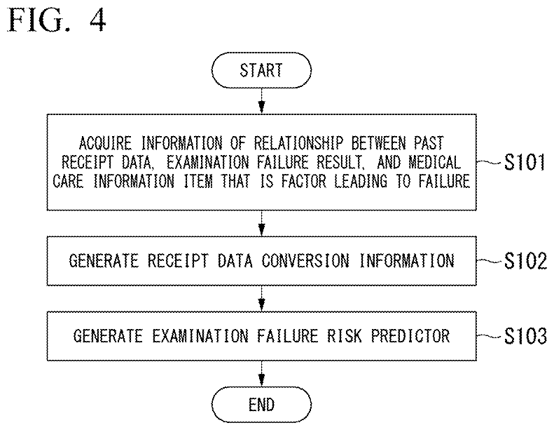

FIG. 4 is a first diagram showing a processing flow of the receipt data examination device according to the first example embodiment.

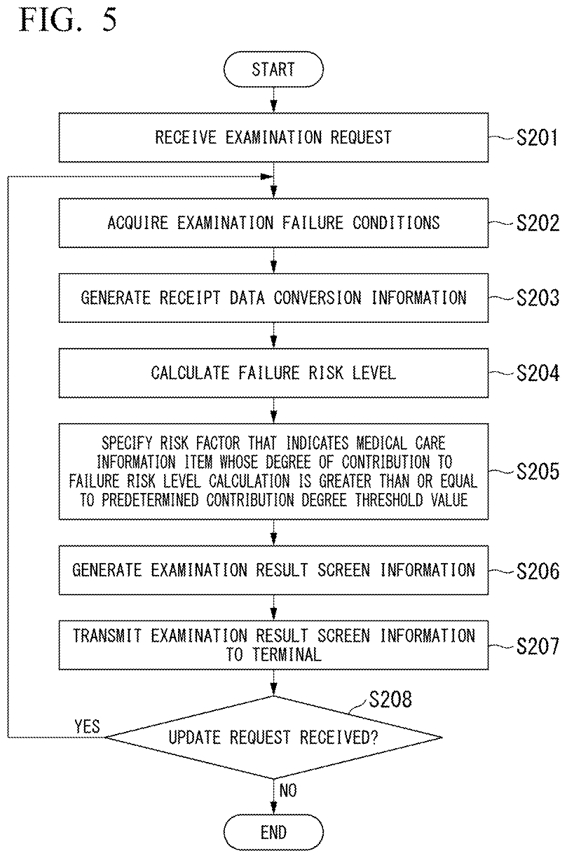

FIG. 5 is a second diagram showing a processing flow of the receipt data examination device according to the first example embodiment.

Next, operations of the receipt data examination device according to the first example embodiment will be described in detail, with reference to FIG. 4 and FIG. 5.

For a plurality of sets of past receipt data, the database 106 stores at least information on the relationship between past receipt data, examination failure results of the receipt data, and medical care information items that are factors of the failures in the examination failure results. The first conversion unit 11 acquires information on the relationship between past receipt data recorded in the database 106 and the examination failure result of the receipt data, and the medical care information item that is a factor of the failure in the examination failure result (Step S101).

The first conversion unit 11 acquires a plurality of examination failure conditions from the database 106. An example of the examination failure conditions includes the relationship between a primary disease name and a plurality of medical care information items such as a medical care method that may possibly be non-compliant with the examination as a medical care method for the primary disease name. The medical care method, which is an example of the medical care information item, includes information such as medication name and medication administration period, for example. The first conversion unit 11 specifies the information of primary disease name “Yes” if the primary disease name recorded in the detailed statement of medical fees indicated by the receipt data matches an examination failure condition. The first conversion unit 11 specifies the information of medication name “Yes” if the primary disease name recorded in the detailed statement of medical fees indicated by the receipt data matches an examination failure condition, and if the medication name matches an examination failure condition. The first conversion unit 11 specifies the information of medication administration period or longer “Yes” if the primary disease name recorded in the detailed statement of medical fees indicated by the receipt data matches an examination failure condition, and if the medication administration period is longer than or equal to that of an examination failure condition as a result of calculating the medication administration period on the basis of the total medication administration amount and the number of daily administrations recorded in the detailed statement of medical fees. As an example, the first conversion unit 11, based on the receipt data, generates receipt data conversion information indicating the primary disease name indicated by an examination failure condition “Yes or No”, the medication name “Yes or No”, and the information on medication period or longer “Yes or No”. In the receipt data conversion information, “Yes” and “No” are held as numerical information such as “1” and “0” respectively. The first conversion unit 11 specifies from the receipt data whether or not there is a record matching a plurality of examination failure conditions, and generates receipt data conversion information that indicates the presence or absence of medical care information items corresponding to a plurality of examination failure conditions (Step S102).

The first conversion unit 11 outputs to the risk predictor generation unit 12 information on the relationship between the receipt data conversion information, the examination failure result of the receipt data used to generate the receipt data conversion information, and the medical care information item that is a factor of the failure in the examination failure result. Based on a lot of receipt data recorded in the database 106, the first conversion unit 11 generates receipt data conversion information, and outputs to the risk predictor generation unit 12 information on the relationship between those pieces of receipt data conversion information and the examination failure results of the receipt data used to generate this information, and the medical care information items that are factors of the failure results in the examination failure results.

The risk predictor generation unit 12 acquires a plurality of pieces of information on relationships between receipt data conversion information and the examination failure results of the receipt data used to generate this information, and the medical care information items that are factors of failure results in the examination failure results. The risk predictor generation unit 12 acquires a plurality of pieces of information on relationships between the acquired receipt data conversion information, the examination failure results, and the medical care information items that are factors of failure results in the examination failure results, and uses machine learning to perform learning processing on these plurality of pieces of information. The risk predictor generation unit 12 performs the learning processing, using methods such as logistic regression and LightGBM. It should be noted that these learning methods are examples, and other methods may be used as long as they enable generation of predictors. The risk predictor generation unit 12 generates an examination failure risk predictor (examination failure risk prediction model) that is a result of machine learning and calculates the failure risk level based on medical care information items included in receipt data (Step S103). It should be noted that the risk predictor generation unit 12 may generate examination failure risk predictors using a plurality of different machine learning methods, and accept a selection of an examination failure risk predictor with the highest accuracy in failure risk level calculation. The risk predictor generation unit 12 stores data of the generated or selected examination failure risk predictors in a storage unit or the like.

With the above processing, the receipt data examination device 1 ends the processing for examining the new examination target receipt data.

In the examination processing, the receipt data examination device 1 then receives, for example, an examination request including receipt data from the terminal 2 (Step S201). The acquisition unit 13 acquires the receipt data included in the examination request. The acquisition unit 13 outputs the receipt data to the second conversion unit 14. The second conversion unit 14 acquires a plurality of examination failure conditions from the database 106 (Step S202). Through the same processing as that of the first conversion unit 11, the second conversion unit 14 generates receipt data conversion information based on examination target receipt data (Step S203).

Specifically, the second conversion unit 14 specifies the information of primary disease name “Yes” if the primary disease name recorded in the detailed statement of medical fees indicated by the receipt data matches an examination failure condition. The second conversion unit 14 specifies the information of medication name “Yes” if the primary disease name recorded in the detailed statement of medical fees indicated by the receipt data matches an examination failure condition, and if the medication name matches an examination failure condition. The second conversion unit 14 specifies the information of medication administration period or longer “Yes” if the primary disease name recorded in the detailed statement of medical fees indicated by the receipt data matches an examination failure condition, and if the medication administration period is longer than or equal to that of an examination failure condition as a result of calculating the medication administration period on the basis of the total medication dosage and the number of daily administrations recorded in the detailed statement of medical fees. As an example, the second conversion unit 14, based on the receipt data, generates receipt data conversion information indicating the primary disease name indicated by an examination failure condition “Yes or No”, the medication name “Yes or No”, and the information on medication period or longer “Yes or No”. The second conversion unit 14 specifies from the receipt data whether or not there is a record matching a plurality of examination failure conditions, and generates receipt data conversion information that indicates the presence or absence of medical care information items corresponding to a plurality of examination failure conditions. The second conversion unit 14 outputs the generated receipt data conversion information to the risk prediction unit 15.

The risk prediction unit 15 acquires an examination failure risk predictor. The risk prediction unit 15 inputs the receipt data conversion information to the examination failure risk predictor, and as a result, calculates a failure risk level (Step S204). Moreover, the risk prediction unit 15 specifies a risk factor indicating a medical care information item with a degree of contribution to failure risk level calculation that is greater than or equal to a prescribed threshold value for degree of contribution (Step S205). If the failure risk level is greater than or equal to a prescribed threshold value, the risk prediction unit 15 may specify a risk factor indicating a medical care information item with a degree of contribution to failure risk level calculation that is greater than or equal to a prescribed threshold value for degree of contribution.

It should be noted that when specifying a risk factor indicating a medical care information item with a degree of contribution to failure risk level calculation that is greater than or equal to a prescribed threshold value for degree of contribution, the risk prediction unit 15 uses a commonly known technique such as correlation analysis to perform calculation. The risk prediction unit 15 outputs to the output unit 16 the calculated failure risk level and the information of the medical care information item that made a significant contribution to the calculation of the failure risk level. The risk prediction unit 15 may calculate the degree of contribution for each of the plurality of medical care information items included in the receipt data conversion information and output it to the output unit 16.

The output unit 16 generates an examination result screen information (revision screen) (Step S206). The output unit 16 may generate an examination result screen information (revision screen) indicating the failure risk level and the name of the medical care information item that made a significant contribution to the calculation of the failure risk level. The output unit 16 may generate an examination result screen information (revision screen) that only indicates the failure risk level if the failure risk level is less than the threshold value. The output unit 16 may calculate the degree of contribution for each of the plurality of medical care information items included in the receipt data conversion information, and generate an examination result screen information (revision screen) displaying the degree of contribution for each medical care information item. If the failure risk level is as low as less than the prescribed threshold value, the output unit 16 may generate an examination result screen information that displays information indicating that the receipt data requires no revision.

The output unit 16 includes, for example, a web server function, and uses this function to transmit the examination result screen information to the terminal 2 (Step S207). The terminal 2 displays the examination result screen information on a web browser or the like. With the above processing, the user that is in charge of medical care administration using the terminal 2 can confirm the numerical value of the failure risk level for the receipt data indicating the created medical fee details, and the name of the medical care information item that made a significant contribution to the calculation of the failure risk level.

According to the processing described above, the receipt data examination device 1 uses an examination failure risk predictor that is generated using a number of pieces of information on the relationship between, past receipt data, examination failure results of the receipt data, and medical care information items that are failure factors in the examination failure results. Therefore, by displaying the failure risk level that is calculated based on cases where records are not incorrect but are deemed non-compliant with the examination according to past cases, such as a case where the relationship between primary disease name and medical care method may possibly be considered inappropriate, in addition to those cases of incorrect records such as simple recording errors, and displaying the name of the medical care information item that made a significant contribution to the calculation of the failure risk level, it is possible to specify not only simple recording errors but also records that may possibly be non-compliant with the examination according to past cases.

The examination result screen information may be an interface screen that allows the user to revise the contents of the medical fee details of the receipt data requested for examination. When the failure risk level is equal to or greater than a threshold value, the user inputs new values and information into the change fields of the receipt data shown by the examination result screen information, for the medical care information item that made a significant contribution to the calculation of the failure risk level. For example, the primary disease name can be changed and the numerical value of the medication dosage can be reduced. The user uses the terminal 2 to input an update instruction to the terminal 2 after the change has been made to the medical care information item. The terminal 2 then transmits an update request including the changed receipt data to the receipt data examination device 1.

The receipt data examination device 1 determines whether an update request has been received (Step S208). If the receipt data examination device 1 has received an update request, the acquisition unit 13 acquires the receipt data included in the update request. Then, upon receiving the update request, the receipt data examination device 1 uses the receipt data included in the update request to perform the above examination processing from Step S202 onward again. The receipt data examination device 1 may automatically prompt the user to transmit an update request, and repeat the examination processing until the failure risk level becomes less than the failure risk level threshold value. This processing is an aspect of the processing in which the receipt data examination device 1 acquires receipt data in which the medical care information item that is the risk factor has been revised on the revision screen, and repeatedly calculates the failure risk level based on the medical care information items included in the receipt data until this failure risk level is less than the threshold value for risk level.

The output unit 16 similarly transmits the examination result screen information to the terminal 2 during the repeated examination processing. The terminal 2 displays the examination result screen information on a web browser or the like. The user confirms the examination result screen information, and saves the receipt data displayed in the examination result screen information, in the terminal 2 if the failure risk level is low. This allows the user to create receipt data with a low failure risk level. Thereafter, the user presents the receipt data with a low failure risk level to the doctor, and receives an instruction to perform revision regarding the receipt data.

According to the first example embodiment described above, the failure risk level of the new examination target receipt data and the risk factor indicating the medical care information item with a degree of contribution to failure risk level calculation that is greater than or equal to a prescribed threshold value for degree of contribution are displayed. According to the processing described above, if the failure risk level is greater than or equal to the prescribed threshold value for risk level, a revision screen for the medical care information item that is a risk factor is outputted, and the revision is repeated until the failure risk level is determined as being low. With the processing described above, the user can use the receipt data examination device 1 to preliminarily select a receipt that is going to fail the examination, and revise the receipt so as to pass the examination, thereby improving the receipt processing duties.

Furthermore, according to the processing described above, the receipt data examination device 1 may sequentially display each medical care information item on the terminal 2 based on the degree of contribution of each medical care information item to the calculation of failure risk level. As a result, it is possible to efficiently revise the problematic portion of receipt data.

Here, the processing of the above receipt data examination device 1 will be described using a specific example of receipt data. It should be noted that the following specific example is an example, and the processing target of the receipt data examination device 1 is not limited to “reflux esophagitis”, and the processing can be performed in a similar manner when using receipt data of other diseases (disease names). For example, receipt data includes records of medication information (medication dosage) and medication dosage, which can be used to calculate that “administration period of XX formulation is 12 weeks”, and injury/disease information such as “reflux esophagitis” as the primary disease name. Based on these medical care information items, the receipt data examination device 1 generates receipt data conversion information indicating the presence or absence of a medical care information item corresponding to an examination failure condition (1 if Yes, 0 if No).

In the case where the presence or absence of the records of medication information (medication administration period) such as “administration period of XX formulation is 12 weeks” and injury/disease information such as “reflux esophagitis” as the primary disease name are indicated by numerical values of 0 and 1, the receipt data examination device 1 can combine these values to thereby create receipt data conversion information that includes a data items indicating 1 if “administration period of XX formulation is 12 weeks and disease is reflux esophagitis” or indicating 0 otherwise.

Using such receipt data conversion information, the risk prediction unit 15 calculates the failure risk level. If the failure risk level is equal to or greater than the threshold value and the risk is high, the output unit 16 outputs the medical care information items that are examination failure factors making a significant contribution to the calculation of the failure risk level. For example, as the examination failure factors, medical care information items that make a significant contribution such as “XX formulation”, “medication administration period, 12 weeks”, and “reflux esophagitis”, that can be interpreted as “administration period of XX formulation is 12 weeks and disease is reflux esophagitis” are displayed as the output result.

Through the revision processing performed by the user, the risk factor revision unit 17 accepts revisions of medical care information items such as “medication administration period, 12 weeks” and “reflux esophagitis” among the medical care information items with high contribution degrees such as “XX formulation”, “medication administration period, 12 weeks”, and “reflux esophagitis”. This means that the records in the receipt data have been revised so that “administration period of XX formulation is 8 weeks and disease is reflux esophagitis” or “administration period of XX formulation is 12 weeks and disease is refractory reflux esophagitis”. The second conversion unit 14 then converts the receipt data revised in such a manner to generate receipt data conversion information, and the risk prediction unit 15 repeats the calculation of the failure risk level.

If the failure risk level is sufficiently low, the receipt data examination device 1 outputs information indicating “No change required”. For example, in the case where the predicted risks of the receipt in which the records have been revised to “administration period of XX formulation is 8 weeks and disease is reflux esophagitis” and the receipt in which the records have been revised to “administration period of XX formulation is 12 weeks and disease is refractory reflux esophagitis” are both sufficiently low, the physician confirms the screen information displaying “No change required” for both of the receipts.

The user, such as a medical care administration clerk, presents the receipt in the case of “No change required” to the physician for confirmation. For example, there is a possibility that either one of the receipt that has been revised to “administration period of XX formulation is 8 weeks and disease is reflux esophagitis” and the receipt that has been revised to “administration period of XX formulation is 12 weeks and disease is refractory reflux esophagitis” is correct. Here, for example, the physician notices a contradiction that XX formulation cannot be prescribed for a period longer than 8 weeks for reflux esophagitis, and if reflux esophagitis is an error and it is supposed to be “refractory reflux esophagitis”, the physician decides the latter of the revised receipts as the receipt with correct records. Through such processing it is possible to prevent examination from being carried out again.

### Second Example Embodiment

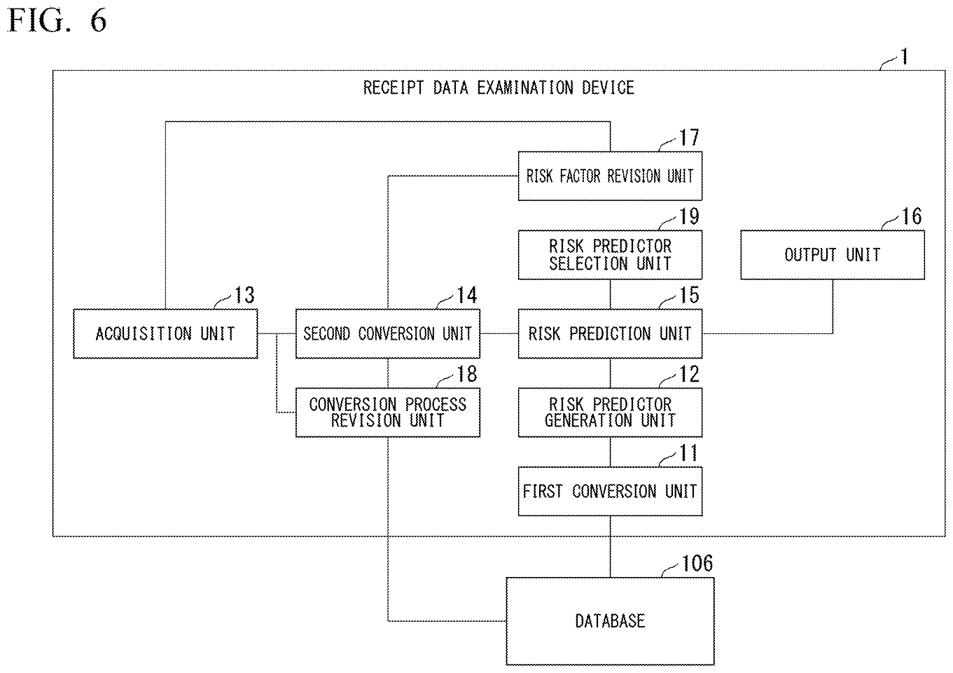

FIG. 6 is a function block diagram of a receipt data examination device according to a second example embodiment.

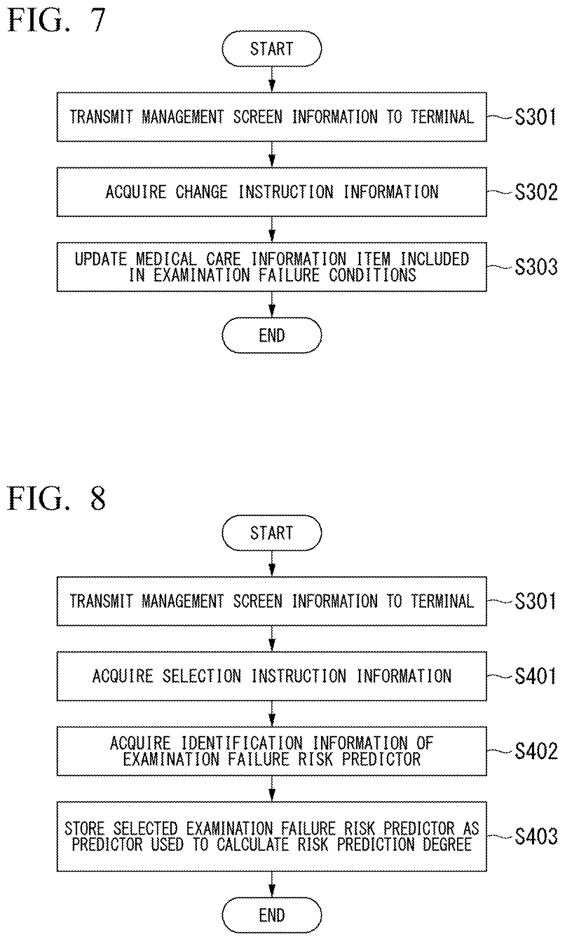

FIG. 7 is a first diagram showing a processing flow of the receipt data examination device according to the second example embodiment.

FIG. 8 is a second diagram showing a processing flow of the receipt data examination device according to the second example embodiment.

Next, operations of the receipt data examination device 1 according to the second example embodiment will be described in detail, with reference to FIG. 6, FIG. 7, and FIG. 8.

The receipt data examination device 1 according to the second example embodiment further includes functions of a conversion process revision unit 18 and a risk predictor selection unit 19 in addition to the functions described with reference to FIG. 4. The conversion process revision unit 18 accepts changes to conditions of medical care information items in the examination failure conditions used to generate receipt data conversion information. The risk predictor selection unit 19 accepts selection of an examination failure risk predictor.

Before the processing of the first example embodiment described above is performed, the conversion process revision unit 18 may accept changes to conditions of medical care information items in the examination failure conditions used to generate receipt data conversion information. Then, the second conversion unit 14 may use the examination failure conditions after the changes have been made to the medical care information items by the conversion process revision unit 18, to convert the receipt data and generate receipt data conversion information.

As an example, the user uses the terminal 2 to access the receipt data examination device 1. The receipt data examination device 1 uses a web server function to transmit management screen information to the terminal 2 (Step S301). The terminal 2 displays the management screen information on the web browser that has been output to a monitor.

On the management screen, the user can specify examination failure conditions, and select and change medical care information items included in the examination failure conditions. For example, the user assumes that an examination failure condition include information on the relationship between the primary disease name and the medical care method inappropriate for the primary disease name. On the management screen, the user changes the information of the medication period in the medical care method under the examination failure condition. The user may be able to perform specification of an examination failure condition, and change such as addition or deletion of medical care information items included in the examination failure condition. The user gives an update instruction by pressing an update button or the like on the management screen. The terminal 2 transmits, to the receipt data examination device 1, the identifier of the examination failure condition and change instruction information including the medical care information items such as medication administration period included in the medical care method to be changed.

The acquisition unit 13 of the receipt data examination device 1 acquires the change instruction information (Step S302). The acquisition unit 13 outputs the change instruction information to the conversion process revision unit 18. The conversion process revision unit 18 reads from the change instruction information the identifier of the examination failure condition, and the medical care information items such as medication administration period included in the medical care method to be changed, and updates the the medical care information items included in the corresponding examination failure condition recorded in the database 106 (Step S303). When performing the examination processing shown in the first example embodiment, in the processing of Step S203 of the receipt data examination device 1, the second conversion unit 14 generates receipt data conversion information using the updated examination failure condition. It should be noted that the first conversion unit 11 may also use the updated examination failure condition to generate receipt data conversion information.

The risk predictor selection unit 19 may include, in the management screen information transmitted to the receipt data examination device 1 when the user accesses the receipt data examination device 1 using the terminal 2, the identifiers of the examination failure risk predictors generated through a plurality of different learning methods, and the accuracy rate, precision rate, recall rate, F-value, ROC curve, AUC (Area Under the Curve), and so forth in the case of using these predictors to perform examination. When the management screen information is displayed on the terminal 2, the identifiers of the examination failure risk predictors generated through the plurality of different learning methods, and the accuracy rate, precision rate, recall rate, F-value, ROC curve, AUC (Area Under the Curve), and so forth in the case of using these predictors to perform examination may be displayed.

The user confirms the identifiers of the examination failure risk predictors generated through the plurality of different learning methods, and the accuracy rate, precision rate, recall rate, F-value, ROC curve, AUC (Area Under the Curve), and so forth that are displayed within the management screen information in the case of using these predictors to perform examination, and specifies one examination failure risk predictor thereamong. The terminal 2 then transmits selection instruction information including the identification information of the specified examination failure risk predictor to the receipt data examination device 1.

The risk predictor selection unit 19 of the receipt data examination device 1 acquires the selection instruction information (Step S401). The risk predictor selection unit 19 acquires the identification information of the examination failure risk predictor selected from the selection instruction information (Step S402). The risk predictor selection unit 19 instructs the examination failure risk predictor to be used by the risk prediction unit 15 to calculate the failure risk level, based on the identification information of the acquired examination failure risk predictor. The risk prediction unit 15 stores the selected examination failure risk predictor as a predictor to be used for calculating the risk prediction level (Step S403). As a result, the risk prediction unit 15 inputs the receipt data conversion information acquired from the second conversion unit 14 to the selected examination failure risk predictor, and calculates the failure risk level.

According to the processing of the second example embodiment described above, it is configured such that the user can change, delete, or add medical care information items included in examination failure conditions as necessary. Therefore, when changes are made to the examination standards at the examination institution, examination failure conditions can be set according to the changes, and new examination failure factors can be added in those cases such as where knowledge of analysts need to be added.

Moreover, according to the processing of the second example embodiment described above, it is configured such that an examination failure risk predictor can be selected based on an evaluation scale, and therefore, calculation of failure risk level can be performed appropriately according to the situation of use.

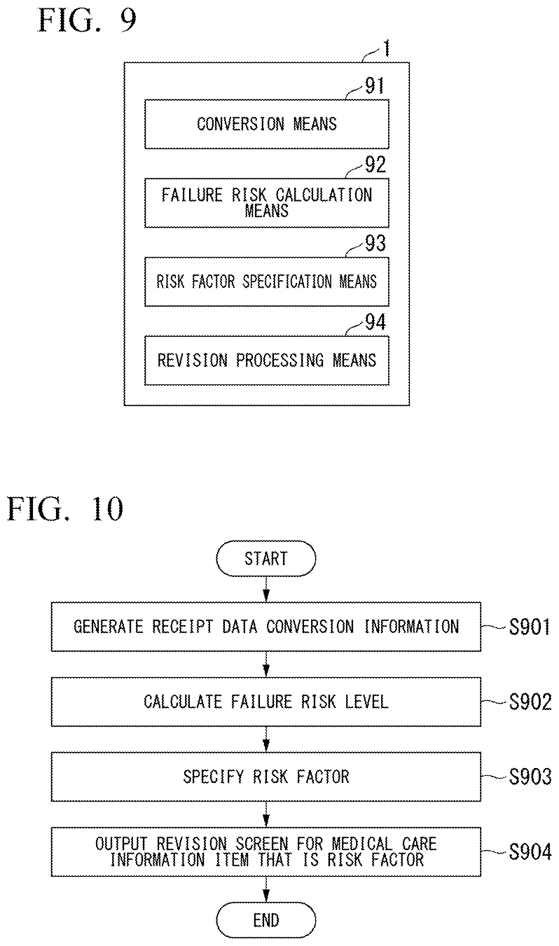

FIG. 9 is a diagram showing a minimum configuration of the receipt data examination device.

FIG. 10 is a diagram showing a processing flow of the receipt data examination device of the minimum configuration.

The receipt data examination device 1 includes at least a conversion means 91, a failure risk calculation means 92, a risk factor specification means 93, and a revision processing means 94.

The conversion means 91 generates, on the basis of acquisition of receipt data indicating a detailed statement of medical fees, receipt data conversion information indicating a presence or absence of medical care information items that correspond to a plurality of examination failure conditions (Step S901).

The failure risk calculation means 92 uses the receipt data conversion information and an examination failure risk predictor to calculate the failure risk level based on the medical care information item included in the receipt data (Step S902).

The risk factor specification means 93 specifies a risk factor indicating a medical care information item with a degree of contribution to failure risk level calculation that is greater than or equal to a prescribed threshold value for degree of contribution (Step S903).

If the failure risk level is greater than or equal to a prescribed threshold value for risk level, the revision processing means 94 outputs a revision screen for the medical care information item that is a risk factor (Step S904).

Each device described above has a built-in computer system. The process of each processing described above is stored in a computer-readable storage medium in the form of a program, and the processing mentioned above is performed by a computer reading and executing the program. Here, the computer-readable storage medium refers to a magnetic disk, a magnetic optical disk, a CD-ROM, a DVD-ROM, a semiconductor memory, or the like. Moreover, the computer program may be distributed to a computer via a communication line, and the computer having received the distributed program may execute the program.

Also, this program may be a program for realizing some of the functions described above. Furthermore, the program may be a so-called difference file (a difference program) which can realize the functions described above in combination with a program already recorded in the computer system.

### DESCRIPTION OF REFERENCE SYMBOLS

1 Receipt data examination device

2 Terminal

11 First conversion unit

12 Risk predictor generation unit

13 Acquisition unit

14 Second conversion unit

15 Risk prediction unit (failure risk calculation means, risk factor specification means)

16 Output unit

17 Risk factor revision unit

18 Conversion process revision unit

19 Risk predictor selection unit

1. A receipt data examination device in communication with a database and a terminal device, the receipt data examination device comprising:
a memory configured to store instructions; and
a processor configured to execute the instructions to:
receive a plurality of examination failure conditions including medical care information items from the database;
accept changes to conditions of the medical care information items included in the plurality of examination failure conditions;
receive an examination request including receipt data from the terminal device, the receipt data indicating a detailed statement of medical fees and including a medical care information item;
generate, based on the receipt data, receipt data conversion information indicating whether a medical care information item is present, using the medical care information items the conditions of which has been changed;
calculate a failure risk level based on the medical care information item included in the receipt data, by using the receipt data conversion information and an examination failure risk predictor;
specify a risk factor indicating a medical care information item with a degree of contribution to a failure risk level calculation that is greater than or equal to a prescribed threshold value for a degree of contribution;
transmit a revision screen for the medical care information item that is a risk factor when the failure risk level is greater than or equal to a prescribed threshold value for risk level to the terminal device; and
if an update request has been received from the terminal device, perform an examination processing again by using receipt data included in the update request.

2. The receipt data examination device according to claim 1, wherein the processor is further configured to execute the instructions to perform learning by machine learning on a relationship between the receipt data stored in past, an examination failure result of the receipt data, and the medical care information item that is a failure factor of the examination failure result, and generate the examination failure risk predictor.

3. The receipt data examination device according to claim 1, wherein the processor is further configured to execute the instructions to output the revision screen for user's decision-making on whether to revise the medical care information item.

4. The receipt data examination device according to claim 1,
wherein the processor is further configured to execute the instructions to:
read, from a change instruction information, an identifier of the examination failure condition, and the medical care information items including medication administration period included in a medical care method to be changed;
update the medical care information items included in the corresponding examination failure condition recorded in the database.

5. The receipt data examination device according to claim 3, wherein the processor is further configured to execute the instructions to use a plurality of different learning processing methods to generate a plurality of examination failure risk predictors for the respective learning processing methods.

6. The receipt data examination device according to claim 5, wherein the processor is further configured to execute the instructions to transmit a management screen information including identifiers of the plurality of examination failure risk predictors generated through the plurality of different learning methods, and an accuracy rate, a precision rate, a recall rate, an F-value, a receiver operating characteristic curve (ROC) curve, and an Area Under a Curve (AUC) in a case of using the plurality of the examination failure risk predictors to perform examination.

7. The receipt data examination device according to claim 6, wherein the processor is further configured to execute the instructions to acquire a selection instruction information including an identification information of the specified examination failure risk predictor.

8. The receipt data examination device according to claim 7, wherein the processor is further configured to execute the instructions to store the selected examination failure risk predictor as a predictor to be used for calculating the risk prediction level.

9. The receipt data examination device according to claim 1, wherein the processor is further configured to execute the instructions to specify, from the receipt data, whether there is a record matching a plurality of examination failure conditions.

10. A receipt data examination method executed by a computer in communication with a database and a terminal device, the method comprising:
receiving a plurality of examination failure conditions including medical care information items from the database;
accepting changes to conditions of the medical care information items included in the plurality of examination failure conditions;
receiving an examination request including receipt data from the terminal device, the receipt data indicating a detailed statement of medical fees and including a medical care information item;
generating, based on the receipt data, receipt data conversion information indicating whether a medical care information item is present, using the medical care information items the conditions of which has been changed;
calculating a failure risk level based on the medical care information item included in the receipt data, by using the receipt data conversion information and an examination failure risk predictor;
specifying a risk factor indicating a medical care information item with a degree of contribution to a failure risk level calculation that is greater than or equal to a prescribed threshold value for a degree of contribution;
transmitting a revision screen for the medical care information item that is a risk factor when the failure risk level is greater than or equal to a prescribed threshold value for risk level to the terminal device; and
if an update request has been received from the terminal device, performing an examination processing again by using receipt data included in the update request.

11. A non-transitory computer readable storage medium storing a program executed by a computer, the program causing the computer to:
receive a plurality of examination failure conditions including medical care information items from the database;
accept changes to conditions of the medical care information items included in the plurality of examination failure conditions;
receive an examination request including receipt data from the terminal device, the receipt data indicating a detailed statement of medical fees and including a medical care information item;
generate, based on the receipt data, receipt data conversion information indicating whether a medical care information item is present, using the medical care information items the conditions of which has been changed;
calculate a failure risk level based on the medical care information item included in the receipt data, by using the receipt data conversion information and an examination failure risk predictor;
specify a risk factor indicating a medical care information item with a degree of contribution to a failure risk level calculation that is greater than or equal to a prescribed threshold value for a degree of contribution;
transmit a revision screen for the medical care information item that is a risk factor when the failure risk level is greater than or equal to a prescribed threshold value for risk level to the terminal device; and
if an update request has been received from the terminal device, perform an examination processing again by using receipt data included in the update request.
